(19) United States
(12) Patent Application Publication
(10) Pub. No.: US 2006/0204511 A1
(43) Pub. Date: Sep. 14, 2006

(54) Use of recombinant or synthetic gelatin as stabiliser in vaccines

(76) Inventors: Jan Bastiaan Bouwstra, Bilthoven (NL); Yuzo Toda, Goirle (NL); Andries Van Es, Dorst (NL)

Correspondence Address: ROGER PITT;KIRKPATRICK & LOCKHART NICHOLSON GRAHAM LLP, 599 LEXINGTON AVENUE, 33RD FLOOR, NEW YORK, NY, 10022-6030, US

(21) Appl. No.: 10/567,078

(22) PCT Filed: Aug. 4, 2004
(86) PCT No.: PCT/NL04/00551

§ 371 Date: Apr. 7, 2006

(30) Foreign Application Priority Data
Aug. 5, 2003 (EP) 030774525

(51) Int. Cl. A61K 39/00

(52) U.S. Cl. 424/185.1

(57) ABSTRACT

The invention relates to methods for preparing vaccine and pharmaceutical compositions comprising recombinant or synthetic gelatin as a stabiliser and to the vaccine and pharmaceutical formulations themselves, as prepared by these methods.

### FIELD OF THE INVENTION

The invention relates to methods for preparing vaccine formulations comprising recombinant or synthetic gelatin as a stabiliser and to the vaccine formulations themselves, as prepared by these methods. In particular, the invention relates to methods for the prevention or delay of crystallisation of recombinant gelatin in vaccine formulations, whereby the stability of the formulation is maintained or increased and the lifetime of the vaccine formulation is maintained or prolonged.

### BACKGROUND OF THE INVENTION

Vaccines are administered to subjects in order to stimulate the immune system and prevent or reduce the severity of subsequent infection with one or more specific microorganism (or infectious agent), such as viruses, bacteria or fungi. The physiologically active substance contained in a vaccine composition may consist of a dead or inactivated microorganism, an attenuated microorganism, a fully virulent organism or one or more antigenic peptides, enzymes, nucleic acid molecules, (monoclonal) antibodies and the like. A major concern in immunization is the stability of the vaccine composition and the World Health Organization issues strict rules for storage of such compositions. Vaccines are known to lose their effectiveness over time, especially when stored under sub-optimal conditions, such as ambient or warm temperatures. As vaccines are often employed in developing countries where optimal storage conditions are difficult to maintain, there is a great need to develop methods by which the ability of the vaccine composition to elicit a protective immune response in a subject is not diminished over time, or is at least diminished as little as possible. For various vaccine compositions great improvements in stability and shelf life have already been achieved. Mainly, two strategies have been employed to improve stability. Firstly, storage at low temperatures (e.g. −10 to −70 degrees Celsius) has been used, but maintenance of such temperatures is often not feasible, especially in developing countries. The second approach has been lyophilization (freeze-drying) and reconstitution to liquid form prior to use. However, also lyophilized vaccine compositions suffer from loss of effectiveness over time. In addition, in both strategies further improvements have been achieved by adding one or more stabilizers to the vaccine compositions.

Vaccine stability is influenced by a number of factors, such as storage temperature, moisture, storage time, the constituents of the composition itself, as well as the surrounding gas and the vial in which the vaccine is kept. As mentioned above, lyophilisation is generally done in the presence of one or more stabilizers in order to improve vaccine stability. Stabilizers used are for example amino acids (such as lysine, arginine, cysteine, monosodium glutamate), detergents, sugars (monosaccharides, disaccharides or polysaccharides), chelating agents, human or bovine serum albumin and various proteins or buffers.

Also gelatin and gelatin derivatives such as hydrolyzed gelatin, or partially hydrolyzed gelatin, have been successfully used as stabilizers in a number of vaccine compositions. Gelatin is a preferred stabilizer because of its known low immunogenicity (see e.g. EP 0 781 779). For example, in EP 568 726 and U.S. Pat. No. 6,039,958 the use of gelatin and hydrolyzed gelatin as a stabilizer of a live vaccine composition containing varicella virus is described. By addition of hydrolyzed gelatin the lyophilized vaccine composition, comprising the normally very heat susceptible virus, retains good effectiveness after storage at 37 degrees Celsius for several weeks. Similarly, DE 3206811 and U.S. Pat. No. 4,555,401 show that gelatin added to liquid, solid or freeze-dried live mumps virus compositions has a positive effect on stability of the vaccine. It is shown that gelatin comprising mumps virus compositions retain effectiveness when stored for several weeks at +20 or −10 degrees Celsius, while compositions lacking gelatin lose effectiveness (measured in a plaque assay).

U.S. Pat. No. 4,147,772 describes the use of partially hydrolyzed gelatin as a stabilizer of a live measles vaccine, whereby the stability of liquid, lyophilized and reconstituted lyophilized compositions was improved. In U.S. Pat. No. 3,859,168 a commercially available gelatin derivative (Haemaccel®) was used as stabilizer in the production of a lyophilized inactivated rabies vaccine.

When using gelatin as a stabilizer, care should be taken that the gelatin solution is made sterile, pyrogen and antigen free. The commercially available gelatin is typically derived from naturally occurring collagen, obtained from hides and bones of animals (especially bovine and porcine sources). A disadvantage of this gelatin is the possibility of immediate hypersensitivity, known as anaphylactic shock. Another disadvantage is the presence of impurities (nucleic acids, proteins, lipids, polysaccharides, etc.) and the fact that the nature of the composition is not clearly defined and thus not reproducible. This may impose additional screening requirements to ensure that the derivation process results in a product with the desired properties and may require careful purification steps. An additional problem posed by gelatin isolated from bovine sources, is the risk of contamination of the gelatin with factors responsible for the occurrence of Bovine Spongiform Encephalitis (BSE) and the new variant of Creutzfeld-Jakob Disease (nvCJD). For this reason the use of gelatin derived from animal sources in pharmaceutical compositions may be prohibited in the future.

WO 01/34801 suggests the use of recombinant gelatin polypeptides as vaccine stabilizer to avoid the above problems associated with the use of gelatin derived from animal sources. However, WO 01/34801 does not actually produce a vaccine composition comprising recombinant gelatin as stabilizer. However, the authors indicate that recombinant gelatin can simply replace the current use of animal-derived gelatin as stabilizer in vaccine compositions, as described above. The present inventors have surprisingly found that such direct replacement is not possible, as recombinant gelatin was found to differ from the so far used animal-derived gelatin. For the use of recombinant gelatin as vaccine stabilizer additional measures have to be taken in order not to compromise vaccine stability and shelf life of the composition.

### SUMMARY OF TH INVENTION

The invention is based on the surprising finding that lyophilized vaccine compositions comprising recombinant or synthetic gelatin as stabiliser were more difficult to produce and had a shorter shelf life compared to analogous compositions comprising animal-derived gelatin. It was found that crystallization occurred in vaccine formulations applying recombinant gelatin as stabilizer during vaccine preparation and storage, whereas such a problem has not been observed with animal-derived gelatin used in the art. A possible explanation could be that animal-derived gelatin is heterogeneous in nature, viz. in view of (amino acid) composition as well as in size. In order to make it feasible to replace animal-derived gelatin with recombinant or synthetic gelatin, the problem of crystallization during vaccine preparation and/or strorage had to be solved. Several methods are provided herein, which enable recombinant or synthetic gelatin to be used as stabilizer in vaccine compositions. Additionally, lyophilized vaccine compositions comprising recombinant or synthetic gelatin are provided, whereby the gelatin in these compositions is not crystallized following the vaccine preparation and/or does not crystallize. Such vaccine compositions are thus suitable for equally long or longer storage times as those suitable for vaccine compositions comprising animal-derived gelatin, with the additional advantages provided by recombinant or synthetic gelatin (purity, etc.).

In one embodiment of the invention a method for preparing a vaccine composition is provided, wherein the vaccine composition comprises recombinant or synthetic gelatin as a stabiliser. The method comprises the step of taking a measure so that the water content of the composition is below 2% following its preparation (e.g. after lyophilization) and/or remains below 2 wt. % during storage in order to prevent the recombinant or synthetic gelatin from crystallisation during the lifetime of the composition.

Further provided is a vaccine composition, particularly a lyophilized vaccine composition, comprising recombinant or synthetic gelatin as a stabiliser, wherein the vaccine composition has a water content of less than 2 wt. %.

In another embodiment of the invention a method for the preparation of a vaccine composition comprising recombinant or synthetic gelatin as a stabiliser is provided, wherein the method comprises the steps of:
(a) producing recombinant or synthetic homodisperse gelatins of various molecular weights,
(b) adding the two or more of these homodisperse gelatins to a vaccine composition as stabiliser and
(c) lyophilizing the vaccine composition, whereby crystallisation of the recombinant gelatin is prevented during the lifetime of the composition.

Also provided is a vaccine composition comprising recombinant or synthetic gelatin as a stabiliser, wherein said gelatin is bi-modal or multi-modal.

### DESCRIPTION OF THE INVENTION

The following definitions are used throughout.

“Recombinant gelatin” (also referred to as recombinant collagen or recombinant collagen-like peptides) as used herein refers to one or more gelatin or gelatin-like polypeptides produced using recombinant methods, such as by expression of a nucleotide sequence encoding the peptide in a microorganism, insect, plant or animal host. Such peptides are characterized by comprising Gly-Xaa-Yaa triplets and by at least 20% of the amino acids being present in the form of consecutive Gly-Xaa-Yaa triplets. Preferably these peptides have a molecular weight of about 2.5 kD or more. In one embodiment the molecular weight of the recombinant gelatin is between about 2.5 and about 50 kD, preferably between about 2.5 and about 30 kD, more preferably between about 2.5 and about 15 kD. In another embodiment the molecular weight of the recombinant gelatin is about 10 kD or less, preferably between about 5 and about 10 kD, more preferably between about 6 and about 8 kD. Recombinant gelatin can be produced as described in EP-A-0926543 and EP-A-1014176 or as in U.S. Pat. No. 6,150,081 incorporated herein by reference.

“Synthetic gelatin” as used herein refers to polypeptides with the same characteristics as recombinant gelatin, except that the peptide is produced by chemical peptide synthesis using known methods. For example, the peptides can be synthesised by the well-known Merrifield solid-phase synthesis method in which amino acids are sequentially added to a growing chain. See Merrifield (1963), J. Am. Chem. Soc. 85:2149-2156; and Atherton et al., “Solid Phase Peptide Synthesis,” IRL Press, London, (1989). Automatic peptide synthesisers are commercially available from numerous suppliers, such as Applied Biosystems, Foster City, Calif.

The recombinant or synthetic gelatin polypeptides used as stabilizers in vaccines may be identical or essentially similar to natural human collagen amino acid sequences (such as for example COL1A1, COL3A1, etc.) or fragments thereof, but also non-human gelatin sequences (such as rat, rabbit, mouse etc.) can be used, or sequences can be designed that do not occur naturally. Such protein sequences, as well as nucleic acid sequences encoding such proteins, are for example described in EP-A-0926543, EP-A-1014176 and WO 01/34646 and are readily available to a skilled person in public sequence databases, such as GenBank, EMBL, SwissProt, etc.

“Animal-derived gelatin” refers to gelatin or gelatin derivatives, such as hydrolyzed or partially hydrolyzed gelatin, obtained from animal tissue, such as hide, bone, skin and the like. Animal-derived gelatin is commercially available.

“Crystallization” as used herein refers to crystallization when using recombinant or synthetic gelatin as vaccine stabilizer compared to animal derived gelatin. Crystallization can occur already during vaccine preparation (e.g. during the lyophilization process) or, alternatively, at a timepoint after preparation, i.e. during storage. The terms “prevention or delay of crystallization” refers on the one hand to the complete prevention of crystallization during the vaccine preparation process, so that at the end of the process a vaccine composition free of crystallized gelatin is provided. On the other hand the terms are used to refer to a prevention or delay of crystallization during subsequent storage of the vaccine composition. For example, crystallization of a vaccine composition comprising recombinant or synthetic gelatin is prevented or delayed if the gelatin comprised in said composition crystallizes not at all, or at the same timepoint or at a later timepoint than the gelatin in analogous composition comprising animal derived gelatin.

The term “lifetime” or “shelf life” as used herein refers to the length of time during which the vaccine composition can be stored without losing its effectiveness in inducing an immune response when administered to a subject. Generally, lyophilized vaccine compositions have a shelf life of one or more months, up to about seven years or more. After this time the compositions have deteriorated too much to be used to confer immunity to a subject. However, shelf life of a vaccine composition depends on various factors, such as the physiologically active substance, the presence of stabilizers (such as gelatin), the tightness of the vial, storage temperature, etc. In order to compare the shelf-life of two vaccine compositions which differ in only one constituent (such as the stabilizer used), these have to be manufactured and stored at identical conditions (or treated identically) and the effectiveness of the compositions has to be compared at various time-points after manufacture (or after specific treatments for testing stability, such as e.g. heating in a waterbath). Two compositions, which differ in only one constituent, are herein referred to as “analogous” compositions.

“Effectiveness” of a lyophilized vaccine composition is used to refer to the ability of the liquid reconstituted composition to evoke the desired immunity when administered to a subject. Effectiveness can be tested by administration to test animals and assessing immunity to infection by the target organism. However, effectiveness does not necessarily have to be tested on live animals. For example, for viral vaccine compositions comprising live or attenuated viral particles, the effectiveness can be assessed using methods, such as the assessment of the viral titer (plaque forming units) after various storage periods or storage conditions.

The inventors made a lyophilized vaccine composition comprising recombinant gelatin (instead of animal-derived gelatin) as stabilizer using methods known in the art. After this composition was stored for some time, it was surprisingly found that crystallization of the composition (and in particular of the gelatin comprised in the composition) had occurred, despite having used the same preparation and storage method as commonly used when making lyophilized vaccine compositions using animal-derived gelatin as stabilizer.

Up to now it was generally assumed that recombinant or synthetic gelatin can directly replace animal-derived gelatin or gelatin derivatives as stabilizers, as suggested in WO 01/34801. Such a replacement would have various advantages, as described above, such as purity (absence of contaminating substances), homogeneity (amino acid sequences and molecular weight distribution can be controlled), safety (no risk of parasites or disease causing prions being present), etc.

The finding by the inventors that recombinant gelatin crystallized easier than animal-derived gelatin when used as stabilizer in lyophilized vaccine compositions indicated that a direct replacement of animal-derived gelatin stabilizers with recombinant gelatin stabilizers brought severe disadvantages with it, namely crystallisation during preparation and crystallization of the vaccine composition upon storage and, therefore, a relative shorter lifetime (or shelf life) of the lyophilized vaccine composition (as a crystallized vaccine composition cannot be used any longer). It was found for example, that a lyophilized recombinant homodisperse gelatin crystallized already during lyophilisation while an equivalent composition comprising animal-derived gelatin crystallized did not show this phenomenon.

In order to use recombinant or synthetic gelatin, rather than an animal-derived gelatin, in vaccine compositions, especially in lyophilized vaccine compositions, the problem of crystallization and the thereby diminished shelf life of the vaccine composition had to be solved. The present invention provides solutions to this problem.

In one embodiment of the invention the problem is solved by ensuring that the water content of the lyophilized vaccine composition is below 2% wt. (weight/volume) after the lyophilization step and by ensuring that the water content constantly remains below 2% (wt.) during the storage of the composition, and preferably until reconstitution and administration to a subject. In a preferred embodiment of the invention the water content of the lyophilized composition is between 1 and 2% and means are employed to keep the water content between 1 and 2% during storage.

A method for preparation and storage of a lyophilized vaccine composition comprising recombinant gelatin is thus provided. This method comprises essentially two steps:
(a) taking a measure so that the water content is below 2 wt. % at the end of the lyophilization process, and
(b) taking a measure to ensure that the water content remains below 2 wt. % during the subsequent storage of the lyophilized composition, in order to prevent or delay crystallization of the recombinant or synthetic gelatin.

Step (a) of the method can be achieved by various means. Using known methods, the lyophilization cycles can be adapted so that the end product has a residual water content below 2 wt. %, preferably between 1 and 2%, more preferably about 1.5%. Water content (residual moisture) of freeze-dried compositions can be measured as described by Greiff (AIChE Symposium Series No. 125, Vol 68, p 7-15). Typically, during the preparation of the lyophilized vaccine the composition (containing all essential components, including the recombinant gelatin stabilizer) is frozen rapidly and the frozen composition is then lyophilized using known methods, as for example described by Greiff et al. (AIChE Symposium Series No. 125, Vol 68, p 7-15) and in U.S. Pat. No. 3,892,876. It is important that the gelatin is frozen in the sol-state and not in the gel-state, because otherwise the lyophilized gelatin will not dissolve again after freeze drying.

To achieve the desired result of having less than 2 wt. % moisture in the freeze-dried composition, it is important that the temperature during freeze-drying constantly remains below the (calculated) glass transition temperature (Tg) of the composition. The calculation method of the glass transition temperature was published by Y. Matveev et. al. in Food Hydrocolloids Vol. 11 no. 2 pp. 125-133, 1997. The importance of the glass transition temperature is well known in the art of freeze drying of formulations containing physiologically active substances, like vaccines. The Tg of compositions can vary greatly, depending on the constituents of the composition, as water content influences Tg. Vice versa, the Tg is influenced by the presence of substances that retain or bind water molecules, such as polar amino acids, sugars, etc.

There are many publications on this subject, for example by Phillips et. al in cryobiology 18, 414419 (1981) or U.S. Pat. No. 801,856. Vaccines like MMR (Mumps Measles Rubella) have in current formulations a critical Tg, which lies around 47 degrees Celsius under dry conditions but rapidly decreases towards room temperature when small amounts of moisture enter the material. Within one week at 37 degrees Celsius a loss in potency of 50% is reported by M. K. Lala in Indian Pediatrics 2003; 40:311-319

Step (b) of the method can be achieved by storing the composition in sealed, air- and/or moisture tight vials or ampoules. Vials or ampoules. as available in the art may be used.

In a preferred embodiment, the vaccine composition is sealed preferably under helium (He), hydrogen (H₂), nitrogen (N₂) or vacuum, less preferably under argon (Ar), least preferably under oxygen (O₂) or carbon dioxide (CO₂). The moisture content of the composition is preferably kept below 2 wt. % until the vaccine is to be administered, preferably for at least 3 months. In another embodiment the moisture content is kept below 2 wt. % for at least 6 months. In a further embodiment of the invention the moisture content is kept below 2 wt. % for at least 1 year or at least 2 years, or at least 7 years.

In a specific embodiment of the invention the recombinant or synthetic gelatin, which is used as stabilizer in a lyophilized vaccine composition, comprises peptides of essentially one specific molecular weight size. Recombinant or synthetic gelatin of essentially one molecular weight size is referred to as “homodisperse”, meaning that at least 90%, preferably at least 95% of the gelatin proteins has a molecular weight that lies within a range of plus or minus 10% around a selected molecular weight. The selected molecular weight is preferably 50 kD or less, such as any molecular weight between 2.5 and 50 kD.

The molecular weight size of recombinant or synthetic gelatin can be measured using methods known in the art, such as SDS-PAGE. It should, however be noted that gelatin peptides migrate according to an apparent molecular weight, which is about a factor 1.4 higher than the true molecular weight (Butkowsky et al. 1982, Meth. Enzymol. 82: 410-423).

When a homodisperse gelatin is added as stabilizer to a vaccine composition, the composition itself is also referred to as being homodisperse. In one embodiment the amino acid sequences of the homodisperse gelatin peptides are “essentially similar” (as defined below).

As applied to the peptides of the invention, the term “essentially similar” means that two peptide sequences, when optimally aligned, such as by the programs GAP or BESTFIT using default parameters, share at least 80 percent sequence identity, preferably at least 90 percent sequence identity, more preferably at least 95 percent sequence identity or more (e.g., 99 or 100 percent sequence identity). GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum 62 (Henikoff & Henikoff, 1992).

In another embodiment of the invention the problem of the invention is solved by using a recombinant or synthetic gelatin as a stabilizer in a lyophilized vaccine composition, whereby the gelatin stabiliser is bi-modal or multi-modal. A “bi-modal gelatin” refers to a composition of two gelatin peptides, each being homodiperse in nature but with different molecular weights. A “multi-modal gelatin” (e.g. tri-modal, tetra-modal, etc.) refers to a composition of more than two gelatin peptides, each being homodiperse in nature but with different molecular weights. The difference in molecular weight prevents crystallization during the vaccine preparation and/or crystallization during subsequent storage. When using a bi-modal gelatin, the molecular weight difference between the two homodisperse gelatins is preferably between 5 kD and 20 kD, most preferably it is about 10 kD. When using a multi-modal gelatin the difference between the homodisperse gelatins is preferably between 3 and 10 kD), most preferably around 5 kD.

A vaccine composition comprising a bi-modal or multi-modal recombinant or synthetic gelatin is also provided herein. Such a vaccine composition solves the problem of crystallization of recombinant or synthetic gelatin during preparation and/or storage.

The problem of the invention is also solved by using a mixture of recombinant or synthetic gelatin peptides of different amino acid sequences, preferably sequences which have less than 95%, preferably less than 90%, more preferably less than 80% amino acid sequence identity, as a stabilizer in a lyophilized vaccine composition.

In still a further embodiment the recombinant or synthetic gelatin polypeptides of the invention are free from helical structure. This is achieved by allowing only partial or preferably no hydroxylation of the proline residues. Partial hydroxylation means that less than 20% of the prolines are hydroxylated, preferably less than 10% or less than 5%. The absence of helical structure prevents gelling of the polypeptides, even at low temperatures. Gelatin free of helix structure can be prepared as disclosed in EP-A-0926543, EP-A-1014176 incorporated herein by reference.

The method of the invention can be used for the preparation and storage of any lyophilized vaccine composition comprising recombinant or synthetic gelatin as stabilizer, irrespective of what the physiologically active component of the vaccine composition is and irrespective of additional stabilizers or other additional components being present. The method can thus be used to prepare a whole range of lyophilized vaccine compositions, such as but not limited to compositions comprising as active component live or attenuated viruses or virus parts (e.g. measels, rubella, mumps, varicella virus, etc.), killed viruses or virus parts, antigenic peptides, DNA vaccines, and so forth. The vaccines may be monovalent vaccines or multivalent (divalent, trivalent, etc.) vaccines. As many different types of vaccines are envisaged, the subject to which the vaccine composition of the invention is administered varies accordingly, and may be a human or an animal, which is to be immunized against a certain infectious agent or autoimmune disease.

In a further embodiment of the invention a vaccine composition comprising recombinant or synthetic gelatin as stabilizer and having a water content of less than 2 wt. % is provided. The vaccine composition can be prepared and stored according to the method of the invention. In particular, the vaccine composition has a shelf life which is at least as long as the shelf life of an analogous composition comprising animal-derived gelatin instead of recombinant or synthetic gelatin. The provided composition has therefore a number of advantages over compositions comprising animal-derived gelatin, but at the same time does not suffer from decreased shelf life. It can be stored for at least three months, or at least 6 months, or at least 1 year or 2 years, or 7 years without losing effectiveness.

The methods of the invention can also be used in the preparation of compositions or formulations other than vaccine compositions. For example, in a further embodiment of the invention the methods developed are used to prepare formulations comprising therapeutic proteins stabilized by recombinant or synthetic gelatin, in order to prevent and/or delay crystallization of the recombinant or synthetic gelatin during preparation and/or storage of the formulations. The methods can be used to prevent or delay crystallization of recombinant or synthetic gelatin during preparation and/or storage of any pharmaceutical composition, such as prophylactic or therapeutic pharmaceutical compositions. The components of the formulations may vary. Suitable formulations are found in Remington's Pharmaceutical Sciences, Mack Publishing Company, Pa, 17^{th} ed. (1985), incorporated herein by reference. The pharmaceutical compositions comprising recombinant or synthetic gelatin as stabilizer and prepared by the methods of the invention are a further embodiment of the invention.

The present invention provides thus methods for preparing vaccine or pharmaceutical compositions comprising recombinant or synthetic gelatin as a stabiliser, wherein measures are taken so that crystallization of said gelatin is prevented during the preparation of the composition (e.g. during the freezing and lyophilization process) and/or during subsequent storage of the vaccine or pharmaceutical composition. These measures for the prevention of crystallization may be selected, without limitation, from one or more of the following: a) adaptation of the freezing and/or lyophilization process (by for example ensuring that the water content of the composition is below 2% at the end of the lyophilization process), b) adaptation of the storage conditions (by for example ensuring that the water content remains below 2% during storage), adaptation of the recombinant or synthetic gelatin used (by for example either using two or more homodisperse gelatins of different molecular weight or by using gelatins with different amino acid sequences).

Provided are also vaccine and pharmaceutical compositions comprising recombinant or synthetic gelatin which is not crystallized following the vaccine preparation and vaccine compositions comprising recombinant or synthetic gelatin which is not crystallized after 3 or 6 or 12 or 24 months of storage, or even 7, 10, 15 or 20 years of storage.

The following non-limiting Examples describe the preparation and storage of lyophilized vaccine compositions comprising recombinant gelatin.

### EXAMPLE 1

Measurement of Gelatin Glass Transition Temperature After Freeze Drying

An aqueous solution of 10% gelatin was made. This solution was quickly frozen in liquid nitrogen and subsequently it was freeze-dried for 48 hours at −55 degrees Celsius. The freeze-dried sample was further dried in a vacuum exsiccator with silicagel. Three different gelatins were prepared using this method. A native alkaline hydrolyzed gelatin (gelatin A) with an average molecular weight of 8 kD and two recombinant gelatins with a molecular weight of 9 kD, of which one recombinant gelatin has a water content above 2% (gelatin B1) and another recombinant gelatin has a water content below 2% (gelatin B2). The water content was controlled by variation of the drying time. Samples with proper water content were selected after measurement of residual water content. The recombinant gelatin was produced according to methods described in EP-A-0926543, EP-A-1014176 and WO01/34646. The sequence of the recombinant gelatin corresponds to the P-monomer as described in EP-A-1014176 Example 1.

DSC (Differential Scanning Calorimetry) was done using a Perkin Elmer DSC 7 instrument under nitrogen atmosphere (flow 20 ml/min). The applied temperature program was:

1 minute hold at 130 degrees Celsius.

130 to 230 degrees Celsius at a heating rate of 5 degrees per minute.

The glass transition temperature was determined according to the half Cp extrapolated method.

Residual moisture amounts were determined by TGA (Thermo Gravimetric Analysis) using a Perkin Elmer TGA 7 under nitrogen atmosphere (flow 20 ml/min).

The applied temperature program was:

1 minute hold at 25 degrees Celsius.

25 to 200 degrees Celsius at a heating rate of 5 degrees per minute.

Residual water content of gelatin A was 2.7%, of gelatin B1 2.4% and of gelatin B2 1.5%.

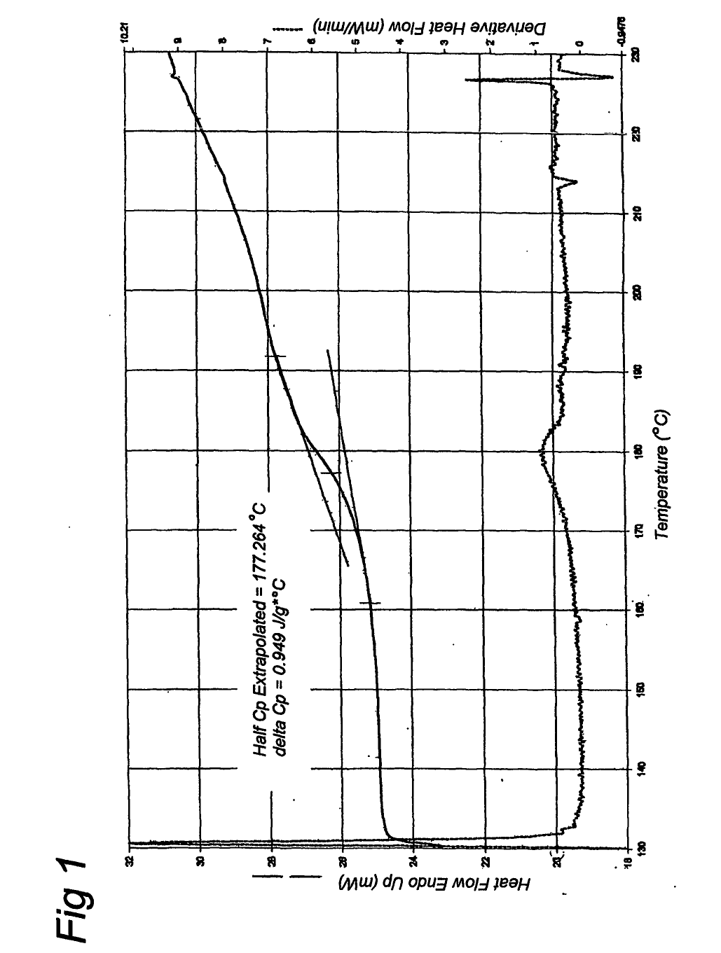

In FIG. 1 the DSC curve of gelatin A is given. A clear glass transition temperature can be seen at a temperature of 177 degrees Celsius. No other phase transitions are observed. This demonstrates that it consists of a complete amorphous glass phase.

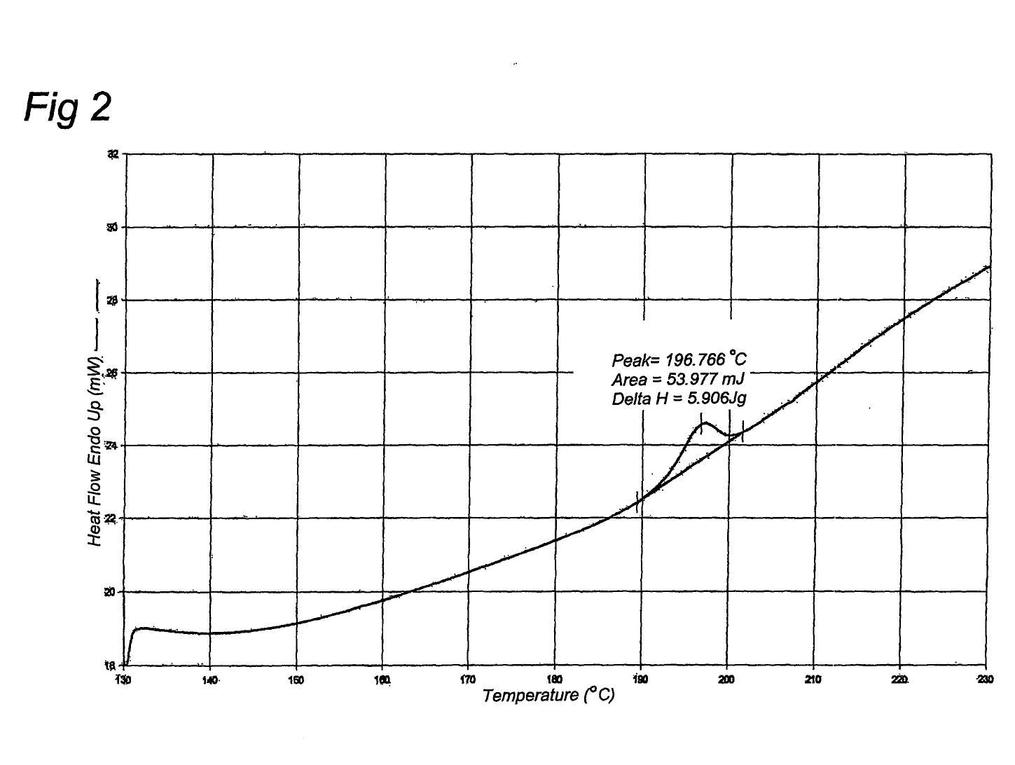

The DSC curve of gelatin B1 is given in FIG. 2. This curve does not show a glass transition temperature. Only a melting peak is seen at 197 degrees Celsius. This clearly demonstrates that crystallization has occurred of the recombinant gelatin during the lyophilization process.

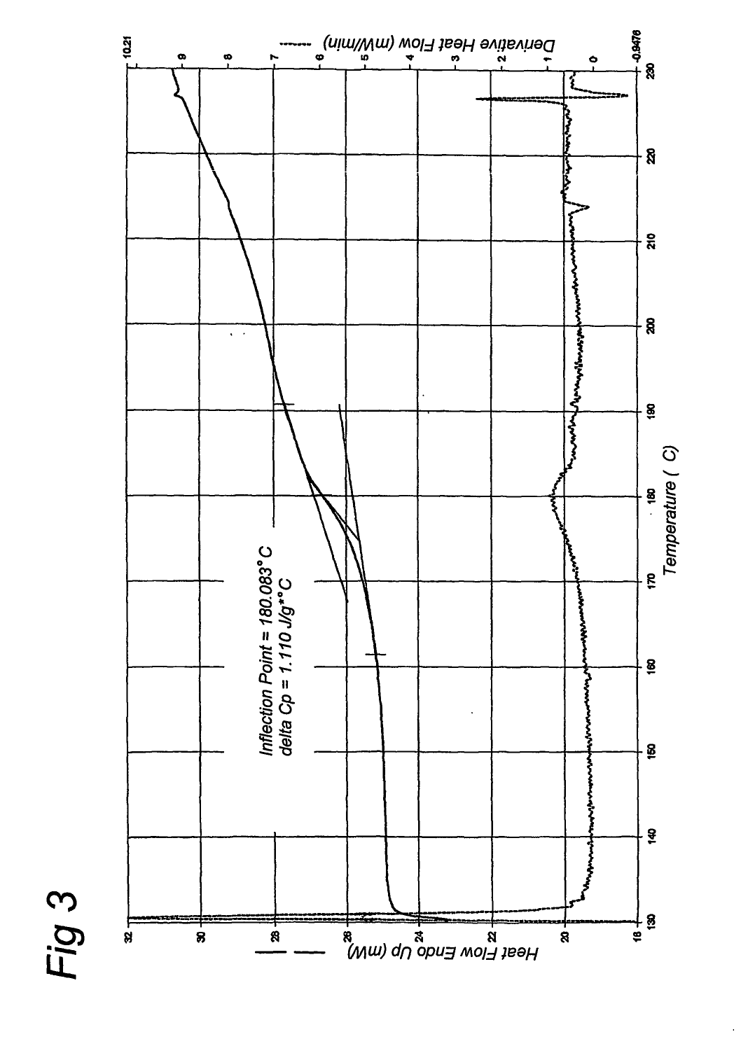

The DSC curve of gelatin B2 is given in FIG. 3. This curve shows a glass transition temperature at 180 degrees Celsius and no melting peak. This demonstrates that when the water content is kept below 2% the crystallization of recombinant gelatin is prevented.

1. Method for the preparation of a vaccine composition comprising recombinant or synthetic gelatin as a stabiliser, said method comprising the step of taking a measure so that the water content remains below 2 wt. % in order to prevent the recombinant gelatin from crystallisation during the lifetime of the composition.

2. Method according to claim 1 in which the recombinant gelatin is homodisperse.

3. Method according to claim 1 in which the molecular weight of the recombinant gelatin is between 2.5 and 50 kD, preferably between 2.5 and 30 kD, and more preferably between 2.5 and 15 kD.

4. Method according to claim 1 in which the molecular weight of the recombinant gelatin is between 5 and 10 kD, preferably between 6 and 8 kD.

5. Method according to claim 1, wherein the amino acid sequences of said gelatin are essentially similar.

6. Method according to claim 1 in which the lifetime is the time from production to the moment of use of the composition.

7. Method according to claim 1 which the lifetime is the period of storage of the composition.

8. Method according to claim 1 which the lifetime is at least 3 months, or at least 6 months, or at least one year or at least 2 years, or at least 7 years.

9. Method according to claim 1 in which the measure that is taken so that the water content remains below 2 wt. % is providing the composition in a sufficiently moisture-tight container.

10. Method according to claim 1 in which the measure that is taken so that the water content remains below 2 wt. % is providing the composition in a sufficiently air-tight container.

11. Vaccine composition comprising recombinant gelatin as a stabiliser, wherein said composition has a water content of less than 2 wt. %.

12. Vaccine composition according to claim 11 which is at least 3 months old.

13. Method for the preparation of a vaccine composition comprising recombinant or synthetic gelatin as a stabiliser, said method comprising the steps of (a) producing recombinant or synthetic bi-modal or multi-modal gelatin, (b) adding said gelatin to a vaccine composition as stabiliser, and (c) lyophilizing said vaccine composition, whereby crystallisation of the recombinant gelatin is prevented during the lifetime of the composition.

14. Vaccine composition comprising recombinant or synthetic gelatin as a stabiliser, wherein said gelatin is bi-modal or multi-modal.

15. Vaccine composition according to claim 14, wherein the amino acid sequences of said gelatin are essentially similar.

16. Method for the preparation of a pharmaceutical composition comprising at least one therapeutic protein and further comprising recombinant or synthetic gelatin as a stabiliser, said method comprising the step of taking a measure so that the water content remains below 2 wt. % in order to prevent the recombinant gelatin from crystallisation during the lifetime of the composition.

17. Pharmaceutical composition comprising at least one therapeutic protein and further comprising recombinant or synthetic gelatin as a stabiliser, wherein said composition has a water content of less than 2 wt. %.

18. Method according to claim 2 in which the molecular weight of the recombinant gelatin is between 2.5 and 50 kD, preferably between 2.5 and 30 kD, and more preferably between 2.5 and 15 kD.

19. Method according to claim 2 in which the molecular weight of the recombinant gelatin is between 5 and 10 kD, preferably between 6 and 8 kD.

20. Method according to claim 2, wherein the amino acid sequences of said gelatin are essentially similar.
